(19) United States
(12) Patent Application Publication
(10) Pub. No.: US 2007/0198033 A1
(43) Pub. Date: Aug. 23, 2007

(54) Peroral Transgastric Endoscopic Techniques

(75) Inventors: Anthony N. Kalloo, Baltimore, MD (US); Sergey Veniaminovich Kantsevoy, Silver Spring, MD (US)

Correspondence Address: FULBRIGHT & JAWORSKI L.L.P., 600 CONGRESS AVE., SUITE 2400, AUSTIN, TX, 78701, US

(73) Assignee: JOHNS HOPKINS UNIVERSITY, BALTIMORE, MD (US)

(21) Appl. No.: 10/580,816

(22) PCT Filed: Nov. 24, 2004
(86) PCT No.: PCT/US04/39663

§ 371 Date: Apr. 11, 2007

Related U.S. Application Data
(60) Provisional application No. 60/525,626, filed on Nov. 26, 2003.
(60) Provisional application No. 60/525,922, filed on Dec. 1, 2003.

(51) Int. Cl. A61B 17/10

(52) U.S. Cl. 606/141

(57) ABSTRACT

Methods and apparatus for accessing the peritoneal cavity and for ligation of fallopian tubes. A representative method includes using an endoscope to orally access a gastric wall. The gastric wall is punctured to provide access to a peritoneal cavity. The endoscope is advanced into the peritoneal cavity through the puncture. A fallopian tube is located and ligated. The endoscope is removed.

This application claims priority to, and incorporates by reference, U. S. Provisional Patent Application Ser. No. 60/525,626, which was filed on Nov. 26, 2003. This application claims also priority to, and incorporates by reference, U. S. Provisional Patent Application Ser. No. 60/525,922, which was filed on Dec. 1, 2003.

### BACKGROUND OF THE INVENTION

1. Field of the Invention

The present invention relates generally to medical techniques and associated devices. More particularly, and in a preferred embodiment, it relates to techniques to prevent pregnancy through new surgical techniques and associated devices. Even more particularly, a preferred embodiment concerns peroral transgastric endoscopic ligation of fallopian tubes.

2. Description of Related Art

Minimally invasive surgery is associated with many proven advantages over traditional open surgery. Laparoscopic access to the peritoneal cavity results in smaller incisions, decreased risk of local and systemic complications, and less postoperative pain with faster recovery. Many abdominal and pelvic surgeries are now being performed laparoscopically, including adrenalectomies, colectomies, gastrectomies, hysterectomies, and tubal ligation.

Nearly 30% of contraceptive users in the United States chose tubal sterilization as their choice in 1995, making tubal ligation one of the most commonly performed surgical procedures. Laparoscopic ligation is associated with decreased operative time, less postoperative pain, shorter hospital stay, and more rapid return to functional activity when compared to traditional minilaparotomy methods.

Traditionally, tubal ligations are performed via a minilaparotomy or a laparoscopic approach. A minilaparotomy is usually done in the postpartum patient and in cases where the surgeon is not trained in laparoscopic methods. It is the preferred technique if patients are morbidly obese or if severe tubal adhesive disease is present; however, it is associated with a higher complication rate, greater need for post-operative analgesia, longer recovery time, and a larger incision when compared to laparoscopy.

While laparoscopic and other techniques may have many advantages as outlined above, they and other more traditional methods require abdominal incisions (laparoscopic or otherwise) in order to provide access to the peritoneal cavity (for example, for ligation of fallopian tubes). However, incision of the skin, subcutaneous fat tissue, and/or abdominal wall muscle can cause numerous potential complications including but not limited to infection, formation of abscess, and post-operative hernias.

It would therefore be advantageous to provide access to the peritoneal cavity without the need for any abdominal incisions, laparoscopic or otherwise.

Referenced shortcomings of conventional methodologies mentioned above are not intended to be exhaustive, but rather are among many that tend to impair the effectiveness of previously known techniques concerning access to peritoneal cavities and, more specifically, access required for ligation of fallopian tubes. Other noteworthy problems may also exist; however, those mentioned here are sufficient to demonstrate that methodology appearing in the art have not been altogether satisfactory and that a significant need exists for the techniques described here.

### SUMMARY OF THE INVENTION

Shortcomings of the prior art are reduced or eliminated by the techniques disclosed here. These techniques are applicable to a vast number of applications, including but not limited to a preferred application involving ligation of fallopian tubes. Other applications involve any procedure that requires access to the peritoneal cavity.

Transgastric endoscopic tubal ligation is less invasive than a laparoscopy and minilaparotomy since it obviates any skin incision, and may be particularly advantageous in morbidly obese patients. The transgastric approach is advantageous because the pelvic organs are a “straight shot” from the gastric cavity requiring minimal endoscopic maneuvering techniques.

In a broad respect, a preferred embodiment concerns a technique of ligation of the fallopian tubes as a method to prevent pregnancy. The technique involves peroral transgastric access to the peritoneal cavity, with localization of the fallopian tubes with application of a ligature with or without subsequent severing of the tubes by cautery or other techniques.

Advantageously, this technique provides peroral transgastric access to the peritoneal cavity without the need for any abdominal incisions, laparoscopic or otherwise.

A large variety of endoscopic accessories can be designed and/or modified for this use, as will be apparent to those having ordinary skill in the art.

In one embodiment, the invention involves a method for ligation of a fallopian tube of a patient. An endoscope is used to orally access a gastric wall. The gastric wall is punctured to provide access to a peritoneal cavity. The endoscope is advanced into the peritoneal cavity through the puncture. The fallopian tube is located. The fallopian tube is ligated, and the endoscope is removed. The method may also include sealing the puncture. Sealing may be done by any method known in the art including sealing by gastric healing. For example, one or more clips may be used. In one embodiment, the clips may be the commercially available Endoclips® by Olympus (Tokyo, Japan). The patient may be human. Puncturing the gastric wall may involve puncturing with a cutter coupled to a dilating balloon, the balloon being inflated to provide access to the peritoneal cavity. Puncturing may involve puncturing with a needle knife electrocautery followed by balloon dilatation with a dilating balloon.

In one embodiment, the invention involves a method of preventing pregnancy including peroral transgastric endoscopic ligation of a fallopian tube of a patient. The patient may be human.

In one embodiment, the invention involves an apparatus for peroral transgastric endoscopic ligation of a fallopian tube of a patient. The apparatus includes an endoscope, a cutter, a dilating balloon, endoscopic forceps, and a first loop. The endoscope is configured to orally access a gastric wall. The cutter is coupled to the endoscope and is configured to puncture the gastric wall. The dilating balloon is coupled to the cutter and is configured to provide access from the puncture into a peritoneal cavity upon inflation. The endoscopic forceps are coupled to the endoscope and are configured to grasp the fallopian tube. The first loop is coupled to the endoscope and is configured to block a patency of the fallopian tube. The apparatus may also include a second loop coupled to the endoscope, the second loop being configured to block a patency of the fallopian tube.

In one embodiment, the involves an apparatus for peroral transgastric endoscopic ligation of a fallopian tube. The apparatus includes an endoscope, a needle knife electrocautery, a dilating balloon, endoscopic forceps, and a first loop. The endoscope is configured to orally access a gastric wall. The needle knife electrocautery is coupled to the endoscope and is configured to puncture the gastric wall. The dilating balloon is coupled to the needle knife and is configured to expand the puncture to provide access into a peritoneal cavity. The endoscopic forceps are coupled to the endoscope and are configured to grasp the fallopian tube. The first loop is coupled to the endoscope and is configured to block a patency of the fallopian tube. The apparatus may also include a second loop coupled to the endoscope, the second loop being configured to block a patency of the fallopian tube.

As used herein, “coupled” is a contextual term that encompasses indirect and direct connections.

Other features and associated advantages will become apparent with reference to the following detailed description of specific embodiments along with the accompanying examples.

### BRIEF DESCRIPTION OF THE DRAWINGS

The following drawings form part of the specification and are included to further demonstrate aspects of embodiments of the invention. The invention may be better understood by reference to one or more of these drawings in combination with the description of illustrative embodiments presented here. The figures are examples only and should not be used to limit the invention. Use of the same element number in the figures indicates identical or similar elements. Drawings are not necessarily to scale.

FIG. 1A is a schematic drawing showing a gastric wall incision with a needle-knife according to embodiments of this disclosure. Note the endoscope inside the sterile overtube.

FIG. 1B is an endoscopic view of a gastric wall incision with the needle-knife according to embodiments of this disclosure.

FIG. 2A is a schematic drawing of a balloon dilatation of the gastric wall according to embodiments of this disclosure.

FIG. 2B is an endoscopic view of balloon dilation of the gastric wall according to embodiments of this disclosure.

FIG. 3A is a schematic drawing of ligation of the uterine tube with Endoloops® according to embodiments of this disclosure.

FIG. 3B is an endoscopic view of uterine tube ligation with Endoloops® according to embodiments of this disclosure.

FIG. 4A is a schematic drawing of a uterine tube legated with two Endoloops®, endoscope withdrawn into the stomach according to embodiments of this disclosure.

FIG. 4B is an endoscopic view of the uterine tube ligated with two Endoloops® according to embodiments of this disclosure.

FIG. 5 is a postmortem examination—ligated fallopian tube, no intra-abdominal infection, abscesses, or adhesions according to embodiments of this disclosure.

### DESCRIPTION OF ILLUSTRATIVE EMBODIMENTS

This disclosure teaches, among other things, a minimally invasive approach to the abdominal and pelvic cavity using a per-oral endoscopic transgastric approach. The transgastric endoscopic approach provides excellent visualization of intra-abdominal and pelvic structures, and the ability to perform therapeutic maneuvers.

Embodiments of this invention can be used in humans for sterilization to prevent unwanted pregnancy. Comparing to existing techniques of surgical or laparoscopic tubal ligation, those embodiments can eliminate incision of the skin, subcutaneous fat tissue and abdominal wall muscle, preventing numerous potential complications including but not limited to: infection, formation of abscess, and post-operative hernias.

In a general embodiment, an upper endoscopy is performed under general anesthesia using sterile technique and equipment. The gastric wall is punctured with an endoscopic balloon or other device suitable to make a puncture and to provide access. If a balloon is used, it is inflated to provide access into the peritoneal cavity. The endoscope is then advanced into the peritoneal cavity. A fallopian tube is located. The tube can be grasped with endoscopic forceps and detachable sterilized Endoloop® applied to the tube to block its patency. In other embodiments, one or more non-reactive silicone bands (Falope Ring Band, Cabot Medical, Langhorne, Pa.) can be used to grab and band a knuckle of the fallopian tube. The endoscope is then be removed, and the gastric wall opening is closed.

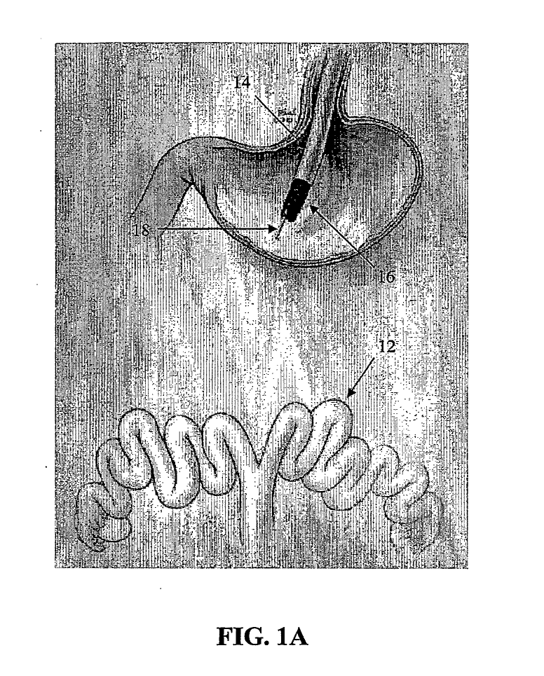

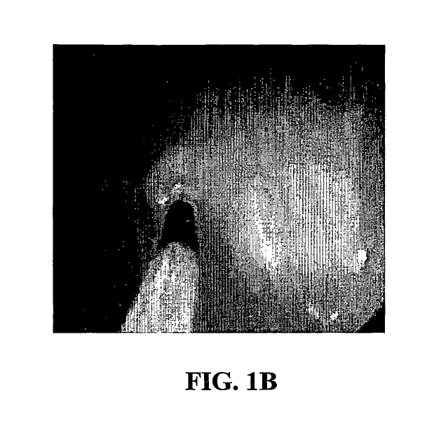

FIGS. 1A-4B illustrate embodiments of this disclosure. FIG. 1A shows an overtube 14, which in one embodiment may be a sterile overtube commercially available from Olympus (Tokyo, Japan). In other embodiments, an overtube may not be present, and in still other embodiments, overtubes of different sizes or configurations may be used. Inside overtube 14 is an endoscope 16. In one embodiment, endoscope 16 may be a forward-viewing, double-channel endoscope such as but not limited to the Olympus GIF-2T160. In other embodiments, a different number of channels or different configuration may be used. A gastric wall incision may be made with a cutter 18. In one embodiment, cutter 18 may be a needle knife electrocautery. In one embodiment, cutter 18 may be a triple lumen, 4 mm cutting-wire needle-knife commercially available from Wilson-Cook Medical Inc. (Winston-Salem, N.C.). In other embodiments, one or more different cutters known in the art may be used. In general, any cutter suitable for forming a gastric opening to a peritoneal cavity may be used. In one embodiment, pure cautery at 20 Joules may be used followed by pure cut at 30 Joules to help achieve access to a peritoneal cavity. In FIG. 1A, the fallopian tube is labeled as element 12. FIG. 1B is an endoscopic view similar to the schematic of FIG. 1A.

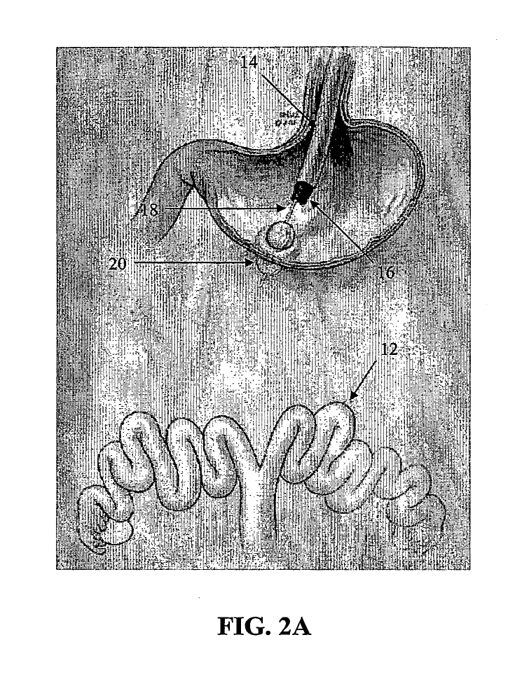

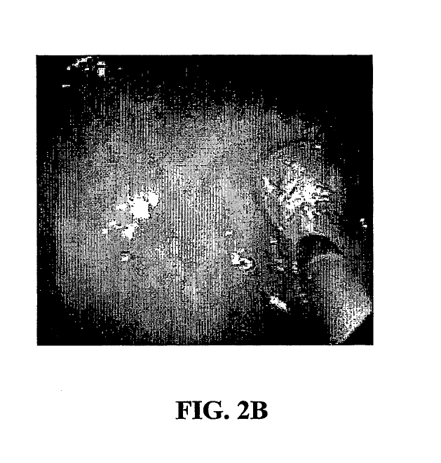

FIG. 2A shows a dilating balloon 20 that is inserted through the gastric incision formed in FIG. 1A. Balloon 20 is coupled to cutter 18. In one embodiment, balloon 20 may be a CRE dilating balloon commercially available from Boston Scientific Microvasive (Natick Mass.). Balloon 20 may be distended according to need (e.g., to accommodate endoscope 16 and/or overtube 14). In one embodiment, balloon 20 may be inserted through the gastric incision and distended to about 20 mm to gain access to the peritoneal cavity. FIG. 2B is an endoscopic view similar to the schematic of FIG. 2A.

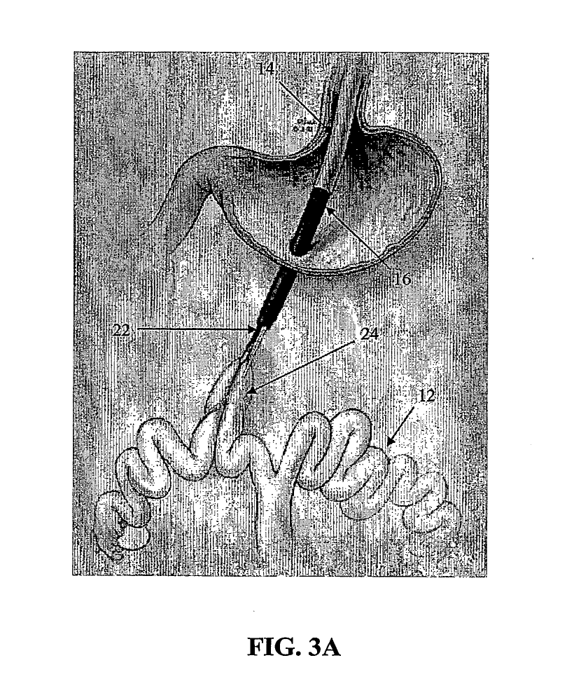

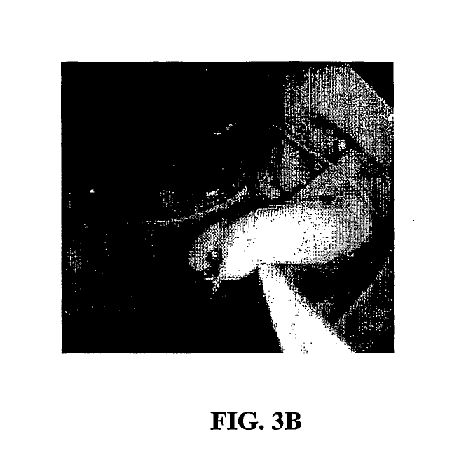

FIG. 3A shows endoscopic forceps 22. Any forceps suitable for grasping a fallopian tube may be used. In one embodiment, forceps 22 are the commercially available Olympus FG-47L1 (Tokyo, Japan). In FIG. 3A, forceps 22 are placed through a loop 24 and then used to gently grasp the fallopian tube 12. In one embodiment, loop 24 may be the commercially available Endoloop® by Olympus (Tokyo, Japan), but any loop suitable for securing and ligating a fallopian tube may be used. For example, in one embodiment, a silicone band may constitute the loop 24 (e.g., the commercially available Falope Ring Band by Cabot Medical (Langhorne, Pa.) may be used). FIG. 3B is an endoscopic view similar to the schematic of FIG. 3A.

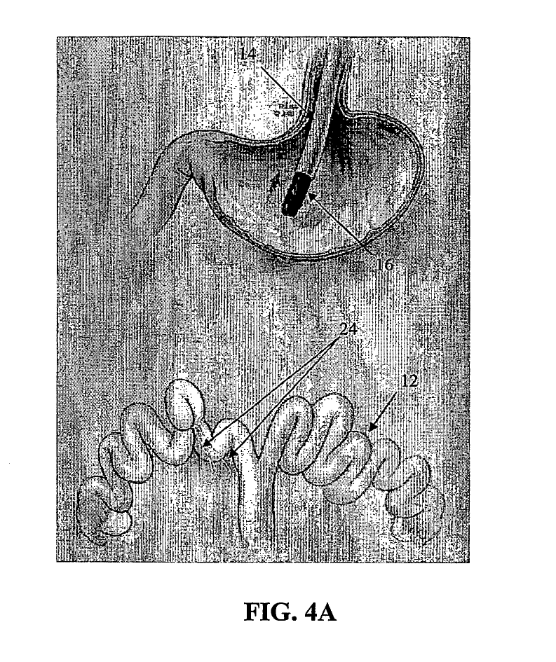

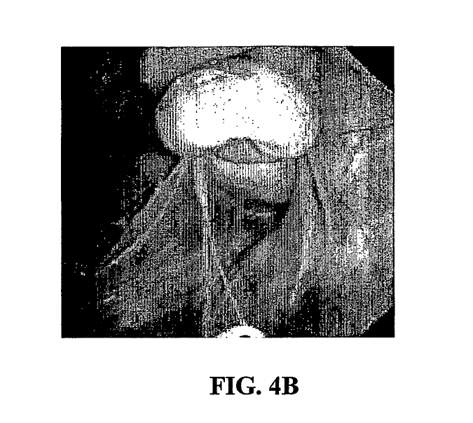

FIG. 4A shows two loops 24 being used for ligation. The two loops may be, in one embodiment, Endoloops® manufactured by Olympus (Tokyo, Japan), but any loops suitable for ligation may be used. FIG. 4B is an endoscopic view similar to the schematic of FIG. 4A.

The techniques of this disclosure are applicable to humans, pigs, or to other animals as will be recognized by those of ordinary skill in the art. The techniques of this disclosure are further described in the example section below. The examples form part of this detailed description.

In different embodiments, procedures other than tubal ligation may be performed. Specifically, techniques of this disclosure can represent the first step taken to enter the peritoneal cavity, for a wide range of surgical and GYN procedures.

In one embodiment, under general anesthesia using sterile technique and equipment, an upper endoscopy is performed. The endoscope can be advanced into the stomach and is insufflated with air. The anterior wall of the abdomen can be trans-illuminated. It is at this site that the wall may be punctured with a needle, under direct view through the endoscope. A guidewire may be advanced through this needle and captured with endoscopic forceps and pulled through a biopsy channel. A sphincterotome may be advanced over the guidewire, into the stomach where the incision into the peritoneal cavity is made.

In another embodiment, an endoscope may be advanced into the stomach and is distended with air (through the endoscope). The anterior abdominal wall is trans-illuminated and is punctured with a needle under direct visualization through the endoscope. A guidewire may be passed into the stomach through the lumen of the needle and captured with endoscopic forceps, then withdrawn through a biopsy channel of the endoscope. A sphinctertome may be placed over the wire and advanced into the stomach, and the incision of the gastric wall may then be made.

Such techniques may be used as an entry for various abdominal surgeries and can eliminate incision of the skin, subcutaneous fat tissue and abdominal wall muscle, preventing numerous potential complications: infection, formation of abscess, post-operative hernias.

Those having ordinary skill in the art will recognize, with the benefit of this disclosure, that other embodiments relating to the general embodiment described above can be used to likewise provide access to, and perform different procedures on associated structures in, the peritoneal cavity.

Additionally, with the benefit of the present disclosure, those having ordinary skill in the art will comprehend that techniques described here may be modified and applied to a number of additional, different applications, achieving the same or a similar result. The attached claims cover all such modifications that fall within the scope and spirit of this disclosure.

The following examples are included to demonstrate specific embodiments of this disclosure. It should be appreciated by those of skill in the art that the techniques disclosed in the examples represent techniques discovered by the inventors to function well in the practice of the invention, and thus can be considered to constitute specific modes for its practice. However, those of ordinary skill in the art should, in light of the present disclosure, appreciate that many changes can be made in the specific embodiments which are disclosed and still obtain a like or similar result without departing from the spirit and scope of the invention.

### EXAMPLE 1

Per-Oral Transgastric Endoscopic Ligation of Fallopian Tubes with Long-Term Survival in a Porcine Model

### BACKGROUND

The inventors have previously reported the safety and feasibility of the per-oral transgastric endoscopic approach for various interventions on intra-abdominal organs (diagnostic peritoneoscopy, liver biopsy, gastrojejunostomy). This approach eliminates incision of abdominal wall providing an alternate approach to diagnostic and therapeutic laparoscopy. This example reports the successful performance of per-oral endoscopic transgastric ligation of fallopian tubes.

Methods

Five 50 kg pigs had general anesthesia and irrigation of the stomach with antibiotic solution. Gastric puncture was performed with a prototype needle knife electrocautery (Olympus®) followed by balloon dilatation of the tract with a biliary dilating balloon (Microvasive). A standard upper endoscope that had high level disinfection followed by gas sterilization was advanced into the peritoneal cavity. Both Fallopian Tubes were identified and one of them was ligated using Olympus Endoloop® with the other serving as a control. Tubal patency was evaluated by hysterosalpingogram in all pigs before and after ligation. All pigs were survived for 1-3 weeks then sacrificed for postmortem examination.

Results

Uterine tubes were easily identified and ligated in all 5 pigs. In each pig fluoroscopy confirmed complete obstruction of ligated tube with preserved patency of the opposite tube. All pigs survived well and ate heartily without any ill-effects. Postmortem examination did not reveal any peritonitis or intra-abdominal adhesions. The Endoloops® were in place with complete obstruction of the ligated tubes.

### CONCLUSION

The endoscopic transgastric approach provides effective ligation of the fallopian tubes in accordance with long-term survival. The endoscopic transgastric approach to the peritoneal cavity may be used in a wide array of diagnostic and therapeutic procedures.

### EXAMPLE 2

Per-Oral Transgastric Endoscopic Ligation of Fallopian Tubes with Long-Term Survival in a Porcine Model

Methods

This study was approved by the Johns Hopkins Animal Care Institutional Review Board. One acute experiment was performed where the pig underwent transgastric endoscopic tubal ligation and was immediately euthanized. Subsequently, transgastric endoscopic tubal ligation was performed on five consecutive 50 kg female pigs (Sus scrofus domesticus). These five pigs were followed post-operatively for 2-3 weeks before euthanization and post-mortem examination.

Pig Preparation:

Six 50 kg pigs were prepared for transgastric endoscopic tubal ligation. The pigs were fed eight 16 oz cans of Ensure (Abbott Laboratories, North Chicago, Ill.) for two days prior to the endoscopic procedure. All procedures were performed under 1.5-2% isoflurane (Abbott Laboratories, North Chicago, Ill.) general anesthesia with 7.0 mm endotracheal intubation (Mallinckrodt Co., C. D. Juarez, Chih, Mexico). Pre-anesthesia medication consisted of an intramuscular injection of 100 mg/mL Telazol (Tiletamine HCl+Zolazepam HCl; Lederle Parenterals, Inc.; Carolina, Puerto Rico) reconstituted with 100 mg/mL Ketamine HCl (Phoenix Pharmaceutical Inc., St. Joseph, Md.) and 100 mg/mL Xylazine (Phoenix Pharmaceutical Inc., St. Joseph, Md.) at a total does of about 0.05 cc/kg. Intramuscular injection of 600,000 units of Penicillin G Benzathine+Penicillin G Procaine (G. C. Hanford Mfg. Co., Syracuse, N.Y.) based antibiotic and 1 gm intravenous Cefazolin (American Pharmaceutical Pterners, Inc., Schaumberg, Ill.) was administered prior to endoscopy. An IV was placed in the marginal ear wein, and Thiopental Sodium (1 gm; Abbott Laboratories, North Chicago, Ill.) was injected at a dose of 6.6-8.8 mg/kg IV.

A 16 Fr Foley catheter with 30 cc Balloon (Rusch Inc., Duluth, Ga.) was inserted into the vagina and the balloon was inflated. 60% Hypaque (Diatrizoate Meglumine Injection, USP) contrast (Amersham Health Inc., Princeton, N. J.) was injected as a baseline hysterosalpingogram was obtained.

Surgical Technique:

All equipment underwent high-level disinfection (Cidex OPA, Ethicon, Inc., Irvine, Calif.) and gas sterilization with ethylene oxide. The antibiotic solution Biobiotic [(Neomycin (40 mg)+Polymyxin B sulfate (2 HTU); diluted in 1 L of saline, Johns Hopkins Pharmacy, Baltimore, Md.) was irrigated in the gastric lumen. Using aseptic technique, a sterile overtube (Olympus, Tokyo, Japan) was placed into the gastric lumen with a forward-viewing double-channel endoscope (Olympus GIF-2T160) inside the overtube. Gastric wall incision was made with a triple lumen 4 mm cutting-wire needle-knife (Wilson-Cook Medical Inc., Winston-Salem, N.C.) using pure cautery at 20 Joules followed by pure cut at 30 Joules (Valleylab SSE2L; Tyco Healthcare Group LP, Boulder, Colo.). FIGS. 1A and 1B illustrate this.

The CRE dilating balloon (Boston Scientific Microvasive, Natick Mass.) was inserted through the gastric incision and distended to 20 mm to gain access to the peritoneal cavity. FIGS. 2A and 2B illustrate this.

The endoscope was advanced into the pelvic cavity and both fallopian tubes were identified. Grasping forceps (Olympus FG-47L-1; Tokyo, Japan) were placed through an open Endoloop® (Olympus, Tokyo, Japan), and then used to gently grasp the fallopian tube. FIGS. 3A and 3B illustrate this.

Two Endoloops® (Olympus Loop, Tokyo, Japan) were placed on a single fallopian tube using identical methods. FIGS. 4A and 4B illustrate this.

A post-ligation hysterosalpingogram was obtained. The endoscope was withdrawn into the gastric lumen while suctioning air from the peritoneal cavity, and the procedure was completed.

Post Operative Period:

All pigs were extubated and recovered within 2-4 hours after the procedure. The pigs were evaluated daily by the investigators for signs of infection. Feedings were resumed on post-operative day #1. After the follow-up period of 2-3 weeks, the pigs were euthanized using identical methods of anesthesia. Necropsy examination was performed. Samples of the ligated fallopian tube were histologically evaluated.

Results:

Six pigs underwent transgastric endoscopic tubal ligation. All pigs had successful ligation performed of one fallopian tube, the other intact tube served as a control. The post-operative recovery and the survival period were without any adverse events. There was no evidence of infection in any pigs during the follow-up, and all pigs ate heartily beginning with post-operative day #1. The operative time from gastric wall incision to completion of the surgery was approximately 20-25 minutes. There were no complications related to deployment and/or placement of the Endoloops®.

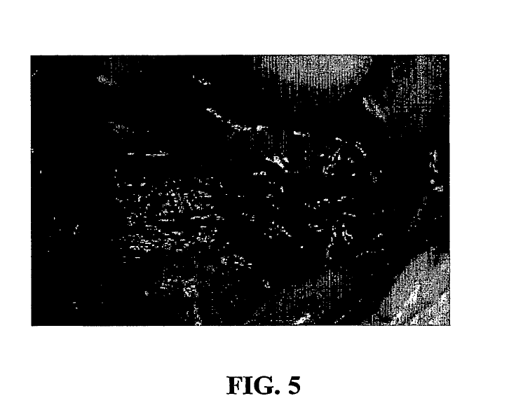

For each pig, the corresponding non-ligated fallopian tube served as a control. Follow-up hysterosalpingogram revealed complete obstruction at the point of ligation with absence of flow of contrast beyond this point, in all pigs. Post-mortem examination did not reveal any evidence of intra-abdominal infection, abscesses, or adhesions. FIG. 5 demonstrates the ligated fallopian tube after 2 weeks of follow-up. Histopathologic examination of the ligated fallopian tube showed chronic inflammatory infiltrates without abscesses.

Discussion

Endoscopic visualization of the pelvic anatomy during the procedures discussed above was superb and identification of the structures was simple. The investigators did not observe any side effects or complications over a 2-3 week follow-up period. All pigs thrived post-surgery. Pre and post-procedure hysterosalpingograms demonstrated complete obstruction of the ligated fallopian tube, and post-mortem pathology revealed obliteration of the lumen in the ligated tube with chronic inflammation and no evidence of abscesses.

This study has demonstrated that the per-oral transgastric approach to tubal ligation is feasible and safe in at least a porcine model with long-term survival of the host.

### REFERENCES

Each of the following references is incorporated by reference in its entirety:
1. Kalloo A N, Kantsevoy S V, Singh V K, Magee C A, Vaughn C A, Hill S L. Flexible transgastric peritoneoscopy: a novel approach to diagnostic and therapeutic interventions in the peritoneal cavity. Gastroenterology 2000; 118: A1039.
2. Gandhi S G, Komenaka I K, Naim J H. Fitz-Hugh-Curtis syndrome after laparoscopic tubal ligation. A case report. J Reprod Med 2003; 48:302-5.
3. Semm K. [Endoscopic intraabdominal surgery in gynecology]. Wien Klin Wochenschr 1983; 95:353-67.
4. Chiou C F, Trussell J, Reyes E, et al. Economic analysis of contraceptives for women. Contraception 2003; 68:3-10.
5. Beger H G, Schwarz A, Bergmann U. Progress in gastrointestinal tract surgery: the impact of gastrointestinal endoscopy. Surg Endosc 2003; 17:342-50.
6. So J B, Chiong E C, Chiong E, et al. Laparoscopic appendectomy for perforated appendicitis. World J Surg 2002; 26:1485-8.
7. Rassweiler J, Seemann O, Schulze M, Teber D, Hatzinger M, Frede T. Laparoscopic versus open radical prostatectomy: a comparative study at a single institution. J Urol 2003; 169:1689-93.
8. Ortega J, Sala C, Garcia S, Lledo S. Cost effectiveness of laparoscopic vs. open adrenalectomy: small savings in an expensive process. J Laparoendosc Adv Surg Tech A 2002; 12:1-5.
9. Muckleroy S K, Ratzer E R, Fenoglio M E. Laparoscopic colon surgery for benign disease: a comparison to open surgery. JSLS 1999; 3:33-7.
10. Kelly J J, Meyers W C, Sandor A, Litwin D E. Laparoscopic gastrectomy in the porcine model: our initial experience with a new hand-assist device. Surg Laparosc Endosc 1999; 9:49-52.
11. Querleu D, Cosson M, Parmentier D, Debodinance P, Crepin G. The Impact of Laparoscopic Surgery on Vaginal Hysterectomy. J Am Assoc Gynecol Laparosc 1994; 1:S29.
12. Farquhar C M, Steiner C A. Hysterectomy rates in the United States 1990-1997. Obstet Gynecol 2002; 99:229-34.
13. Sherman P A, Burigo J A. Comparison of laparoscopic Falope-Ring and minilaparotomy sterilization. Obstet Gynecol 1984; 63:71-5.
14. Westhoff C, Davis A. Tubal sterilization: focus on the U.S. experience. Fertil Steril 2000; 73:913-22.
15. Kalloo A N, Singh V K, Niiyama H, et al. Flexible Transgastric Peritoneoscopy: A Novel Approach to Diagnostic and Therapeutic Interventions in the Peritoneal Cavity. Gastrointest Endosc; 2004; 114-117.
16. Penfield A J. Minilaparotomy for female sterilization. Obstet Gynecol 1979; 54:184-8.
17. ACOG practice bulletin. Benefits and risks of sterilization. Number 46, September 2003 (replaces Technical Bulletin Number 222, April 1996). Int J Gynaecol Obstet 2003; 83:339-50.
18. Poliakoff S R, Yoon I B, King T M. A four year experience with the Yoon Ring. In: Phillips J M, ed. Endoscopy in Gynecology. American Association of Gynecologic Laparoscopists. Downey, Calif., 1978.
19. Atabek U, Mayer D, Amin A, Camishion R C. Pancreatic cystogastrostomy by combined upper endoscopy and percutaneous transgastric instrumentation. J Laparoendosc Surg. 1993 October; 3(5):501-4.
20. Molloy D, Kaloo P D, Cooper M, Nguyen T V. Laparoscopic entry: a literature review and analysis of techniques and complications of primary port entry. Aust N Z J Obstet Gynaecol. 2002 August; 42(3): 246-54.
21. Jacobson M T, Osias J, Bizhang R, Tsang M, Lata S, Helmy M, Nezhat O, Nezhat C. The direct trocar technique: an alternative approach to abdominal entry for laparoscopy. JSLS. 2002 Apr-Jun; 6(2): 169-74.
22. Mellinger J D, Ponsky J L. Percutaneous endoscopic gastrostomy: state of the art, 1998. Endoscopy. 1998 February; 30(2): 126-32.
23. Sadler D J, Gray R R, So C B, Saliken J C. A simplified method of gastropexy for transgastric enterostomy. Clin Radiol. 1999 July; 54(7):462-4.

1. A method for ligation of a fallopian tube of a patient, comprising:
using an endoscope to orally access a gastric wall;
puncturing the gastric wall to provide access to a peritoneal cavity;
advancing the endoscope into the peritoneal cavity through the puncture;
locating the fallopian tube;
ligating the fallopian tube; and
removing the endoscope.

2. The method of claim 1, further comprising sealing the puncture.

3. The method of claim 1, where the patient is human.

4. The method of claim 1, where puncturing the gastric wall comprises puncturing with a cutter coupled to a dilating balloon, the balloon being inflated to provide access to the peritoneal cavity.

5. The method of claim 1, where puncturing the gastric wall comprises puncturing with a needle knife electrocautery followed by balloon dilatation with a dilating balloon.

6. A method of preventing pregnancy comprising peroral transgastric endoscopic ligation of a fallopian tube of a patient.

7. The method of claim 6, where the patient is human.

8. An apparatus for peroral transgastric endoscopic ligation of a fallopian tube of a patient, comprising:
an endoscope configured to orally access a gastric wall;
a cutter coupled to the endoscope and configured to puncture the gastric wall,
a dilating balloon coupled to the cutter and configured to provide access from the puncture into a peritoneal cavity upon inflation;
endoscopic forceps coupled to the endoscope and configured to grasp the fallopian tube; and
a first loop coupled to the endoscope and configured to block a patency of the fallopian tube.

9. The apparatus of claim 8, further comprising a second loop coupled to the endoscope and configured to block a patency of the fallopian tube.

10. An apparatus for peroral transgastric endoscopic ligation of a fallopian tube, comprising:
an endoscope configured to orally access a gastric wall;
a needle knife electrocautery coupled to the endoscope and configured to puncture the gastric wall;
a dilating balloon coupled to the needle knife and configured to expand the puncture to provide access into a peritoneal cavity;
endoscopic forceps coupled to the endoscope and configured to grasp the fallopian tube; and
a first loop coupled to the endoscope and configured to block a patency of the fallopian tube.

11. The apparatus of claim 10, further comprising a second loop coupled to the endoscope and configured to block a patency of the fallopian tube.
